(19) United States
(12) Patent Application Publication
(10) Pub. No.: US 2011/0079522 A1
(43) Pub. Date: Apr. 7, 2011

(54) MULTI-ANALYTE TEST STRIP WITH INLINE WORKING ELECTRODES AND SHARED OPPOSING COUNTER/REFERENCE ELECTRODE

(75) Inventors: Graeme WEBSTER, Inverness (GB); Marco F. Cardosi, Croy (GB); Selwayan Saini, Culbokie (GB)

(73) Assignee: LifeScan Scotland Limited, Inverness (GB)

(21) Appl. No.: 12/572,534

(22) Filed: Oct. 2, 2009

(51) Int. Cl. G01N 27/26; G01N 33/50

(52) U.S. Cl. 205/792; 204/403.01

(57) ABSTRACT

A co-facial multi-analyte test strip includes a first insulating layer with an electrically conductive layer disposed thereon. The electrically conductive layer includes a first working electrode with a first analyte contact pad and a second working electrode with a second analyte contact pad. In addition, the first and second working electrodes of the electrically conductive layer are disposed on the first insulating layer in a planar inline configuration. The multi-analyte test strip also includes a patterned spacer layer positioned above the electrically conductive layer, with the patterned spacer layer defining a single bodily fluid sample-receiving chamber therein that overlies the first working electrode and the second working electrode. The multi-analyte test strip further includes a shared counter/reference electrode layer overlying and exposed to the bodily-fluid sample receiving chamber and configured in an opposing relationship to the first and second working electrodes; and a second insulating layer disposed above the shared counter/reference electrode layer. Moreover, the co-facial multi-analyte test strip also has a multi-analyte reagent layer disposed on the electrically conductive layer with the multi-analyte reagent layer having a first analyte reagent portion disposed on the first working electrode within the sample-receiving chamber and a second analyte reagent layer disposed the second working electrode within the sample-receiving chamber.

### BACKGROUND OF THE INVENTION

1. Field of the Invention

The present invention relates, in general, to medical devices and, in particular, to analyte test strips, test meters and related methods.

2. Description of Related Art

The determination (e.g., detection and/or concentration measurement) of an analyte in a fluid sample is of particular interest in the medical field. For example, it can be desirable to determine glucose, ketones, cholesterol, acetaminophen and/or HbA1c concentrations in a sample of a bodily fluid such as urine, blood or interstitial fluid. Such determinations can be achieved using analyte test strips, based on, for example, photometric or electrochemical techniques, along with an associated test meter.

Typical electrochemical-based analyte test strips employ a working electrode along with an associated counter/reference electrode and enzymatic reagent to facilitate an electrochemical reaction with a single analyte of interest and, thereby, determine the concentration of that single analyte. For example, an electrochemical-based analyte test strip for the determination of glucose concentration in a blood sample can employ an enzymatic reagent that includes the enzyme glucose oxidase and the mediator ferricyanide. Such conventional analyte test strips are described in, for example, U.S. Pat. Nos. 5,708,247; 5,951,836; 6,241,862; and 6,284,125; each of which is hereby incorporated in full by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified perspective, exploded depiction of a co-facial multi-analyte test strip according to an embodiment of the present invention;

FIGS. 2A-2F are simplified top views of the first insulating layer, electrically conductive layer, multi-analyte reagent layer, patterned spacer layer, shared counter/reference electrode layer and second insulating layer, respectively, of the co-facial multi-analyte test strip of FIG. 1;

FIG. 3 is a simplified top view of the co-facial multi-analyte test strip of FIG. 1;

FIG. 4 is simplified depiction of the electrically conductive layer and shared counter/reference electrode layer of a co-facial multi-analyte test strip according to an embodiment of the present invention in use with a test meter also according to an embodiment of the present invention; and

FIG. 5 is a flow diagram depicting stages in a process for determining multiple analytes in a single bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Co-facial (also sometimes referred to as “opposing electrode”, “opposing” or “opposed”) multi-analyte test strips according to embodiments of the present invention include a first insulating layer with an electrically conductive layer disposed thereon. The electrically conductive layer includes a first working electrode with a first analyte contact pad and a second working electrode with a second analyte contact pad. In addition, the first working electrode and the second working electrode of the electrically conductive layer are disposed on the first insulating layer in a planar inline configuration.

The co-facial multi-analyte test strips also include a patterned spacer layer positioned above the electrically conductive layer, with the patterned spacer layer defining a single bodily fluid (e.g., whole blood) sample-receiving chamber therein that overlies the first working electrode and the second working electrode. The multi-analyte test strips further include a shared counter/reference electrode layer overlying and exposed to the single bodily-fluid sample receiving chamber and configured in an opposing (i.e., co-facial) relationship to the first and second working electrodes. The shared counter/reference electrode has a counter/reference electrode contact pad.

The co-facial multi-analyte test strips also have a second insulating layer disposed above the shared counter/reference electrode layer. Moreover, the co-facial multi-analyte test strips have a multi-analyte reagent layer disposed on the electrically conductive layer with the multi-analyte reagent layer having a first analyte reagent portion (such as a glucose reagent portion) disposed on the first working electrode within the bodily fluid sample-receiving chamber and a second analyte reagent portion (e.g., a ketone reagent portion) disposed the second working electrode within the bodily fluid sample receiving chamber.

Co-facial multi-analyte test strips according to the present invention are beneficial in that a plurality of non-identical analytes (e.g., the analyte glucose and the ketone analyte 3-hydroxybutyrate) can be determined in a single bodily fluid sample (such as a single whole blood sample). In addition, since the co-facial multi-analyte test strips include a shared counter/reference electrode layer (i.e., a counter/reference electrode that is used during the determination of both the first analyte and the second analyte), the co-facial multi-analyte test strips, and their bodily fluid sample-receiving chamber, are beneficially small in size. Moreover, the use of an inline configuration for the first working electrode and second working electrode enables the co-facial multi-analyte test strips to employ a straight sample chamber with straight bodily fluid sample flow, thus simplifying manufacturing and operation of the co-facial multi-analyte test strip. Furthermore, since the shared reference/counter electrode layer is configured in an opposing (i.e., co-facial) configuration with respect to the first working electrode and the second working electrode, the sample-receiving chamber has a beneficially small volume and the overall test strip can be beneficially compact.

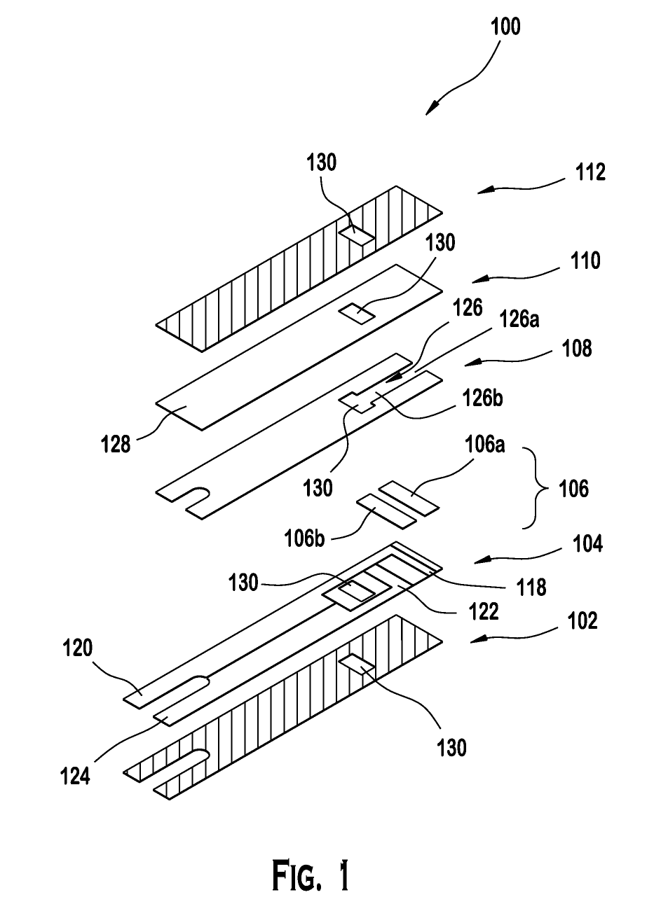

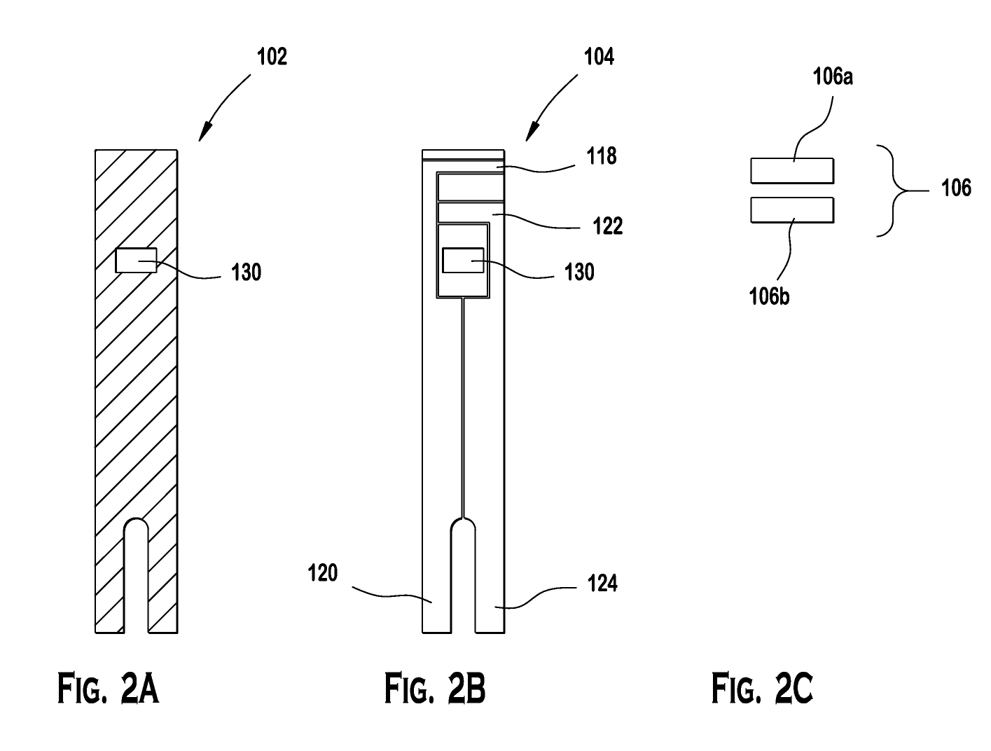

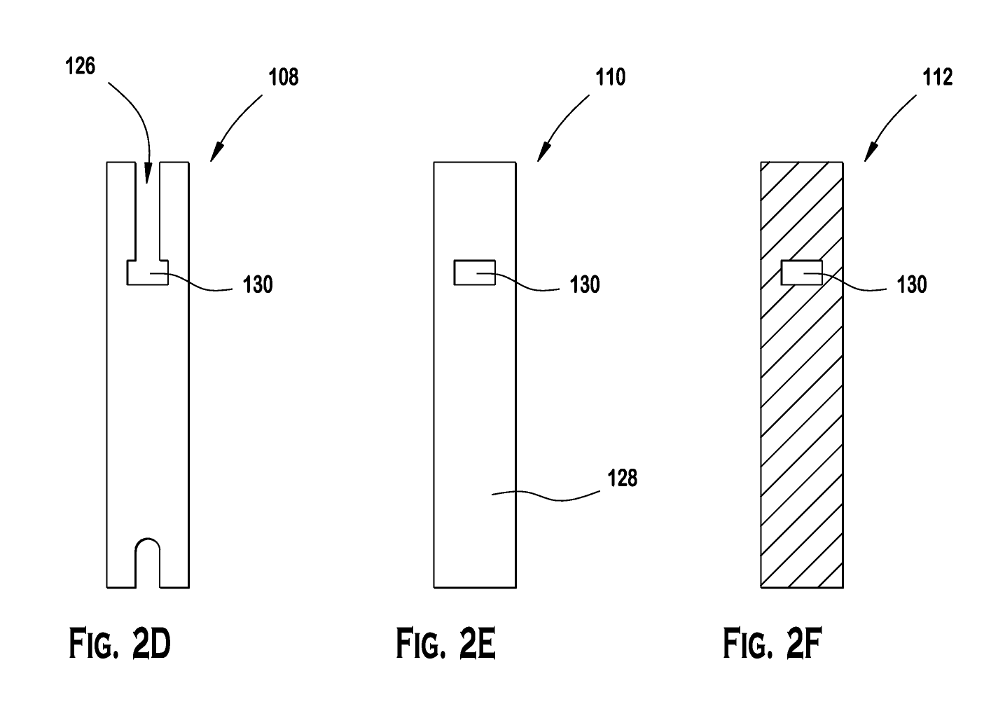

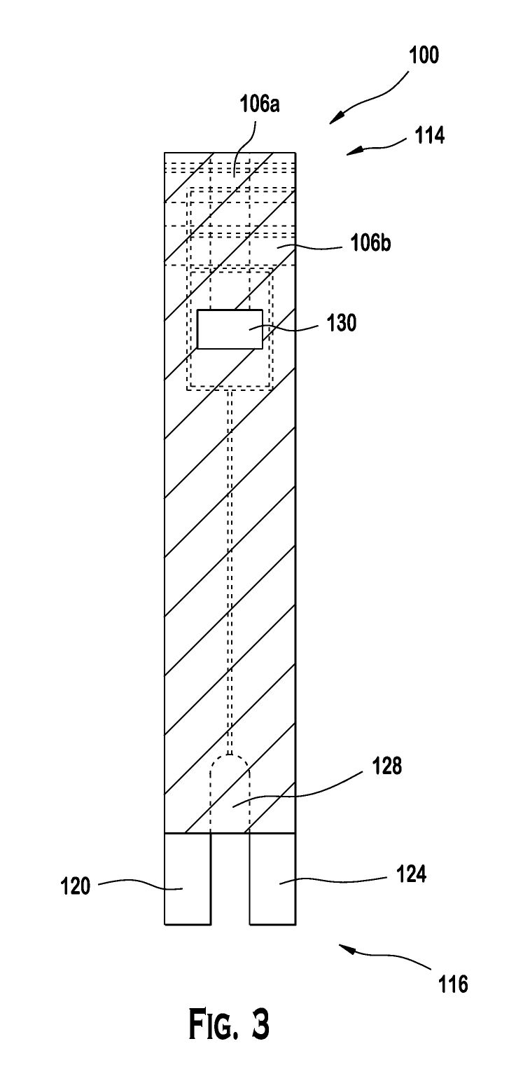

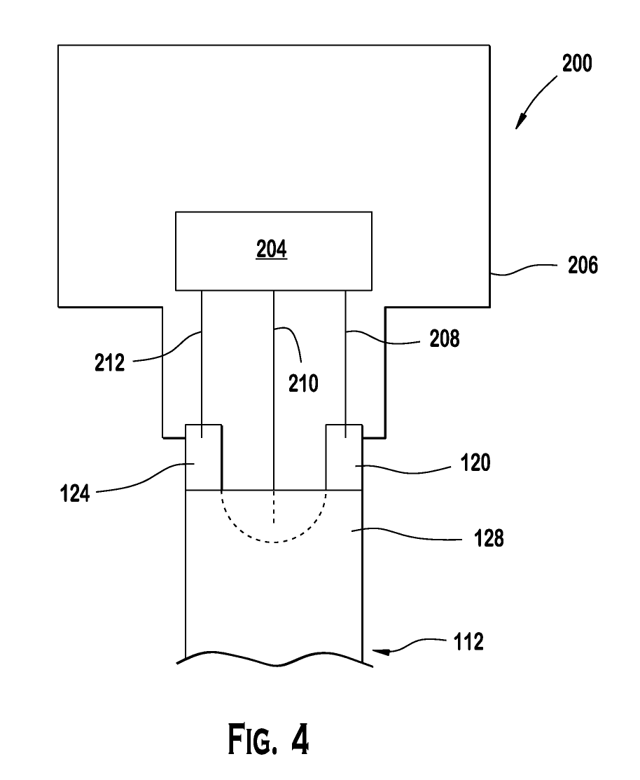

FIG. 1 is a simplified perspective, exploded depiction of a co-facial multi-analyte test strip 100 according to an embodiment of the present invention. FIGS. 2A-2F are simplified top views of a first insulating layer 102, electrically conductive layer 104, multi-analyte reagent layer 106, patterned spacer layer 108, shared counter/reference electrode layer 110 and second insulating layer 112, respectively, of co-facial multi-analyte test strip 100. FIG. 3 is a simplified top view of co-facial multi-analyte test strip 100.

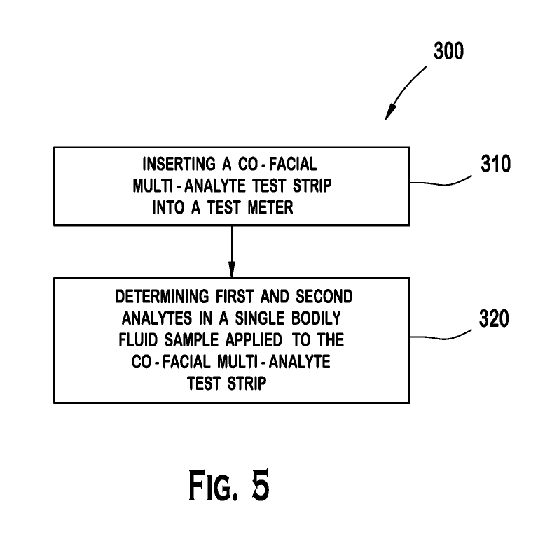

Referring to FIG. 1, FIGS. 2A through 2F and FIG. 3, co-facial multi-analyte test strip 100 is configured for use with a test meter (described further herein, for example with respect to the embodiment of FIG. 4) and includes a proximal end 114 and a distal end 116 (see FIG. 3). Co-facial multi-analyte test strip 100 also includes first insulating layer 102, with electrically conductive layer 104 disposed thereon.

Electrically conductive layer 104 includes a first working electrode 118 with a first analyte contact pad 120 and a second working electrode 122 with a second analyte contact pad 124 (see FIG. 2B in particular). Patterned spacer layer 108 of co-facial multi-analyte test strip 100 is disposed above electrically conductive layer 104 (see FIG. 1 in particular), with the patterned spacer layer defining a single bodily fluid (e.g., whole blood) sample-receiving chamber 126 therein that overlies first working electrode 118 and second working electrode 122. In addition, single bodily fluid sample-receiving chamber 126 has a proximal end 126a and a distal end 126b.

Shared counter/reference electrode layer 110 overlies, and is exposed to, single bodily fluid sample-receiving chamber 126 and is configured in an opposing relationship to first working electrode 118 and second working electrode 122. In addition, shared counter/reference electrode has a counter/reference electrode contact pad 128.

Second insulating layer 112 of co-facial multi-analyte test strip 100 is disposed above shared counter/reference electrode layer 110, as depicted in FIG. 1.

Co-facial multi-analyte test strip 100 includes multi-analyte reagent layer 106 disposed on electrically conductive layer 104 (see FIG. 1 in particular). Multi-analyte reagent layer 106 has a first analyte reagent portion 106a (such as a glucose reagent portion) disposed on first working electrode 118 within single bodily fluid sample-receiving chamber 126 and a second analyte reagent portion 106b (e.g., a ketone reagent portion) disposed on second working electrode 122 within single bodily fluid sample-receiving chamber 126.

In the embodiment of FIGS. 1, 2A-2F and 3, first analyte contact pad 120, counter/reference electrode contact pad 128, and second analyte contact pad 124 are configured for contact with electrical connector pins of a test meter. Moreover, the first working electrode 118 and second working electrode 122 are disposed on first insulating layer 102 in a planar inline configuration beneath single bodily fluid sample-receiving chamber 126. Therefore, a bodily fluid sample applied to proximal end 126a of single bodily fluid sample-receiving chamber 126 will be conducted down the single bodily fluid sample-receiving chamber 126 and across first working electrode 118 and subsequently across second working electrode 120 since the first and second working electrodes are in-line with one another with respect to the direction of bodily fluid sample flow.

Co-facial multi-analyte test strip 100 also includes a vent opening 130 that extends entirely through first insulating layer 102, electrically conductive layer 104, patterned spacer layer 108, shared counter/reference electrode layer 110 and second insulating layer 112. Vent opening 130 is in fluidic communication with distal end 126b of single bodily fluid sample-receiving chamber 126 and facilitates the conduction of a bodily fluid sample down the single bodily fluid sample-receiving chamber 126 by capillary forces. Moreover, vent opening 130 is further configured as a stop flow junction at distal end 126b of the single bodily fluid sample-receiving chamber 126. Such a stop flow junction is configured to prevent a bodily fluid sample from exiting distal end 126b of single bodily fluid sample-receiving chamber 126 due to an abrupt change in fluid flow cross-section at the intersection of vent opening 130 and distal end 126b.

The manufacturing of co-facial multi-analyte test strip 100 is beneficially simplified by having vent opening 130 extend entirely through co-facial multi-analyte test strip 100. For example, vent opening 130 can be created using a conventional and simple punch processing after first insulating layer 102, electrically conductive layer 104, patterned spacer layer 108, shared counter/reference electrode layer 110 and second insulating layer 112 have been assembled. Such punch processing also eliminates the need to align individual vent openings created in each of the first insulating layer 102, electrically conductive layer 104, patterned spacer layer 108, shared counter/reference electrode layer 110 and second insulating layer 112. In addition, such a vent opening is open to the test strip surroundings at the outer surface of both the first and second insulating layers, thus increasing vent reliability via redundancy. However, once apprised of this disclosure, one skilled in the art will recognize that co-facial multi-analyte test strips according to embodiments of this invention can include a vent opening that provides fluidic communication between distal end 126 and the external surface of either first insulating layer 102 or second insulating layer 112.

First insulating layer 102 and second insulating layer 112 can be formed, for example, of a suitable plastic (e.g., PET, PETG, polyimide, polycarbonate, polystyrene), silicon, ceramic, or glass material. For example, the first and second insulating layers can be formed from a 7 mil polyester substrate.

In the embodiment of FIGS. 1, 2A-2F and 3, first working electrode 118 and shared counter/reference electrode layer 110, along first analyte reagent portion 106a, are configured to electrochemically determine a first analyte concentration in a bodily fluid sample (such as glucose in a whole blood sample) using any suitable electrochemical-based technique known to one skilled in the art. Furthermore, second working electrode 122 and shared counter/reference electrode layer 110, along with second analyte reagent portion 106b, are configured to electrochemically determine a second analyte concentration in the same bodily fluid sample (such as the ketone 3-hydroxybutyrate).

First electrically conductive layer 104 can be formed of any suitable conductive material such as, for example, gold, palladium, carbon, silver, platinum, tin oxide, iridium, indium, or combinations thereof (e.g., indium doped tin oxide). Moreover, any suitable technique can be employed to form first electrically conductive layer 104 including, for example, sputtering, evaporation, electro-less plating, screen-printing, contact printing, or gravure printing. For example, first electrically conductive layer 104 can be a sputtered palladium layer with the first and second working electrodes defined via laser ablation.

Shared counter/reference electrode layer 110 can, for example, be a gold layer that is sputter coated in the underside of second insulating layer 112 using conventional techniques known in the art. In addition, vent opening 130 can be formed using conventional punching techniques and punch tooling.

Patterned spacer layer 108 serves to bind together first insulating layer 102 (with first electrically conductive layer 104 thereon) and second insulating layer 112 with shared counter/reference electrode layer 110 on the underside thereof. Patterned spacer layer 108 can be, for example, a 95 um thick, double-sided pressure sensitive adhesive layer, a heat activated adhesive layer, or a thermo-setting adhesive plastic layer. Patterned spacer layer 108 can have, for example, a thickness in the range of from about 1 micron to about 500 microns, preferably between about 10 microns and about 400 microns, and more preferably between about 40 microns and about 200 microns.

First analyte reagent portion 106a can be any suitable mixture of reagents known to those of skill in the art that selectively reacts with a first analyte, such as, for example glucose, in a bodily fluid sample to form an electroactive species, which can then be quantitatively measured at the first working electrode of co-facial multi-analyte test strips according to embodiments of the present invention. Therefore, first analyte reagent portion 106b includes at least an enzyme and a mediator. Examples of suitable mediators include, for example, ferricyanide, ferrocene, ferrocene derivatives, osmium bipyridyl complexes, and quinone derivatives. Examples of suitable enzymes include glucose oxidase, glucose dehydrogenase (GDH) using a pyrroloquinoline quinone (PQQ) co-factor, GDH using a nicotinamide adenine dinucleotide (NAD) co-factor, and GDH using a flavin adenine dinucleotide (FAD) co-factor. First analyte reagent portion 106a can be applied using any suitable technique.

Second analyte reagent portion 106b can be any suitable mixture of reagents known to those of skill in the art that selectively reacts with a second analyte such as, for example the ketone 3-hydroxybutyrate, in a bodily fluid sample to form an electroactive species, which can then be quantitatively measured at the second working electrode of co-facial multi-analyte test strips according to embodiments of the present invention. Therefore, second analyte reagent portion 106b includes at least an enzyme and a mediator. Second analyte reagent portion 106b can be applied using any suitable technique. It should be noted that the first and second analytes are dissimilar. In other words, the first and second analytes are not the same chemical species. Therefore, two different analytes are determined by co-facial multi-analyte test strips according to the present invention.

When the second analyte is the ketone 3-hydroxybutyrate, the mediator can be, for example, a mixture of potassium ferricyanide and NAD and the enzyme can be, for example, a mixture of diaphorase and hydroxybutyrate dehydrogenase.

Once apprised of the present invention, one skilled in the art will recognize that first analyte reagent portion 106a and second analyte reagent portion 106b can, if desired, also contain suitable buffers (such as, for example, Tris HCl, Citraconate, Citrate and Phosphate), surfactants (for example, Tritoan X100, Tergitol NP-&, PLuronic F68, Betaine and Igepal), thickeners (including, for example, hydroxyethylcellulose, HEC, carboxymethylcellulose, ethylcellulose and alginate) and other additives as are known in the field.

Test meters for use with a co-facial multi-analyte test strip according to embodiments of the present invention include a test strip receiving module and a signal processing module. The test strip receiving module has a first electrical connector configured for contacting a first analyte contact pad of a first working electrode of the co-facial multi-analyte test strip; a second electrical connector configured for contacting a counter/reference electrode contact pad of a shared counter/reference electrode layer of the co-facial multi-analyte test strip; and a third electrical connector configured for contacting a second analyte contact pad of a second working electrode of the co-facial multi-analyte test strip.

The signal processing module is configured to receive a first electrical signal via the first electrical connector and the second electrical connector and employ that first signal for the determination of a first analyte (such as glucose) in a bodily fluid sample applied to the co-facial multi-analyte test strip. The signal processing module is also configured to receive a second electrical signal via the second electrical connector and third electrical connector and employ the second electrical signal for the determination of a second analyte (such as the ketone 3-hydroxybutyrate) in the bodily fluid sample applied to the co-facial multi-analyte test strip. Furthermore, the first analyte electrode, and second analyte electrode are disposed on a first insulating layer of the co-facial multi-analyte test strip in a planar inline configuration while the shared counter/reference electrode layer is disposed in an opposing (co-facial) configuration with respect to the first and second working electrodes. The signal processing module can be configured to receive the first electrical signal and the second electrical signal sequentially, simultaneously or in a time-overlapping manner.

Test meters according to embodiments of the present invention are beneficially small in size, inexpensive and readily manufactured since they employ a single electrical connector for contacting a counter/reference electrode pad that is used for the determination of multiple analytes. In other words, only three electrical connectors are needed to determine multiple analytes, thus reducing test meter size, cost and manufacturing difficulty.

FIG. 4 is simplified depiction of a first electrically conductive layer 104 and shared counter/reference electrode layer 110 of a co-facial multi-analyte test strip according to an embodiment of the present invention in use with a test meter 200 also according to an embodiment of the present invention (with dashed line indicating features that are hidden from view in the perspective of FIG. 4). Test meter 200 includes a test strip receiving module 202 and a signal processing module 204 within case 206.

Test strip receiving module 202 includes a first electrical connector 208 configured for contacting first analyte contact pad 120 of a first working electrode of the co-facial multi-analyte test strip; a second electrical connector 210 configured for contacting a counter/reference electrode contact pad 128 of a shared counter/reference electrode layer of the co-facial multi-analyte test strip; and a third electrical connector 212 configured for contacting a second analyte contact pad 124 of a second working electrode of the co-facial multi-analyte test strip.

Signal processing module 204 is configured to receive a first signal via first electrical connector 208 and second electrical connector 210 and employ the first signal for the determination of a first analyte in a bodily fluid sample applied to the co-facial multi-analyte test strip. Signal processing module 204 is also configured to receive a second signal via second electrical connector 210 and third electrical connector 212 and employ the second signal for the determination of a second analyte in the bodily fluid sample applied to the co-facial multi-analyte test strip.

In the embodiment of FIG. 4, signal processing module 204 includes, for example, a signal receiving component, a signal measurement component, a processor component and a memory component (each not shown in FIG. 4). Test meter 200 can measure, for example, electrical resistance, electrical continuity or other electrical characteristic between first working electrode 118 and shared counter/reference electrode layer 110 and between second working electrode 122 and shared counter/reference electrode layer 110. One skilled in the art will appreciate that the test meter 200 can also employ a variety of sensors and circuits that are not depicted in simplified FIG. 4 during determination of a first analyte and a second analyte.

FIG. 5 is a flow diagram depicting stages in a method 300 for determining multiple analytes (for example, the analyte glucose and the analyte 3-hydroxybutyrate) in a single bodily fluid sample (such as a whole blood sample) according to an embodiment of the present invention.

At step 310 of method 300, a co-facial multi-analyte test strip is inserted into a test meter. The insertion of the test strip into the meter is such that a first electrical connector of the test meter comes into contact with a first analyte contact pad of a first working electrode of the co-facial multi-analyte test strip; a second electrical connector of the test meter comes into contact with a counter/reference electrode contact pad of a shared counter/reference electrode layer of the co-facial multi-analyte test strip; and a third electrical connector of the test meter comes into contact with a second analyte contact pad of a second working electrode of the co-facial multi-analyte test strip.

The method also includes determining at least a first analyte and a second analyte in a single bodily fluid sample applied to the multi-analyte test strip using a signal processing module of the test meter (see step 320 of FIG. 5). During the determining step, the signal processing module receives a first signal via the first electrical connector and the second electrical connector and employs the first signal for the determination of a first analyte. Also during the determining step, the signal processing module receives a second signal via the second electrical connector and the third electrical connector and employs the second signal for the determination of a second analyte.

Once apprised of the present disclosure, one skilled in the art will recognize that method 300 can be readily modified to incorporate any of the techniques, benefits and characteristics of co-facial multi-analyte test strips according to embodiments of the present invention and described herein, as well as those of test meters according to embodiments of the present invention described herein. Moreover, the bodily fluid sample can be applied to the co-facial multi-analyte test strip either before the inserting step or after the inserting step.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. A co-facial multi-analyte test strip comprising:
a first insulating layer;
an electrically conductive layer disposed on the first insulating layer, the electrically conductive layer including:
a first working electrode with a first analyte contact pad; and
a second working electrode with a second analyte contact pad;
a patterned spacer layer positioned above the electrically conductive layer, the patterned spacer layer defining a single bodily fluid sample-receiving chamber therein that overlies the first working electrode and the second working electrode, the single bodily fluid sample-receiving chamber having a proximal end and a distal end;
a shared counter/reference electrode layer overlying and exposed to the sample receiving chamber, the shared counter/reference electrode layer configured in an opposing relationship to the first working electrode and the second working electrode, the shared counter/reference electrode layer having a counter/reference electrode contact pad; and
a second insulating layer disposed above the shared counter/reference electrode layer;
wherein the co-facial multi-analyte test strip further includes:
a multi-analyte reagent layer disposed on the electrically conductive layer, the multi-analyte reagent layer including:
a first analyte reagent portion disposed on at least a portion of the first working electrode within the sample receiving chamber; and
a second analyte reagent portion disposed on at least a portion of the second working electrode within the sample receiving chamber; and
wherein the first working electrode and second working electrode are disposed on the first insulating layer in a planar inline configuration.

2. The co-facial multi-analyte test strip of claim 1 further including a vent opening in fluidic communication with the distal end of the single bodily-fluid sample receiving chamber.

3. The co-facial multi-analyte test strip of claim 2 wherein the vent opening extends entirely through the first insulating layer, electrically conductive layer, patterned spacer layer, shared counter/reference electrode layer and second insulating layer.

4. The co-facial multi-analyte test strip of claim 2 wherein the vent opening is further configured as a stop flow junction at the distal end of the single bodily fluid sample-receiving chamber.

5. The co-facial multi-analyte test strip of claim 1 wherein the test strip has a test strip proximal end and a test strip distal end and the proximal end of the single bodily fluid sample-receiving chamber is open to the proximal end of the test strip.

6. The co-facial multi-analyte test strip of claim 1 wherein the first analyte reagent portion is nonidentical in comparison to the second analyte reagent portion.

7. The co-facial multi-analyte test strip of claim 5 wherein the first analyte reagent portion is a glucose analyte reagent.

8. The co-facial multi-analyte test strip of claim 6 wherein the second analyte reagent portion is a ketone analyte reagent.

9. The co-facial multi-analyte test strip of claim 7 wherein the ketone is 3-hydroxybutyrate.

10. The co-facial multi-analyte test strip of claim 1 wherein the bodily fluid sample is a whole blood sample.

11. A test meter for use with a co-facial multi-analyte test strip, the test meter comprising:
a test strip receiving module with at least:
a first electrical connector configured for contacting a first analyte contact pad of a first working electrode of the co-facial multi-analyte test strip;
a second electrical connector configured for contacting a counter/reference electrode contact pad of a shared counter/reference electrode of the multi-analyte test strip; and
a third electrical connector configured for contacting a second analyte contact pad of a second working electrode of the multi-analyte test strip; and
a signal processing module,
wherein the signal processing module is configured to receive a first signal via the first electrical connector and the second electrical connector and employ the first signal for the determination of a first analyte in a bodily fluid sample applied to the co-facial multi-analyte test strip; and
wherein the signal processing module is also configured to receive a second signal via the second electrical connector and third electrical connector and employ the second signal for the determination of a second analyte in the bodily fluid sample applied to the co-facial multi-analyte test strip; and
wherein the first electrical connector and third electrical connector are configured for essentially coplanar contact with the first analyte contact pad and the second analyte contact pad and the second electrical contact pad is configured to contact the counter/reference electrode contact pad in a manner offset from the essentially coplanar contact.

12. The test meter of claim 11 wherein the second electrical connector is disposed between the first electrical connector and the third electrical connector.

13. The test meter of claim 11 wherein the first analyte is non-identical in comparison to the second analyte.

14. The test meter of claim 13 wherein the first analyte is glucose.

15. The test meter of claim 14 wherein the second analyte is a ketone.

16. The test meter of claim 15 wherein the ketone is 3-hydroxybutyrate.

17. The test meter of claim 11 wherein the bodily fluid sample is a whole blood sample.

18. The test meter of claim 11 wherein the signal processing module is configured for determination of the first analyte and determination of the second analyte via an electrochemical-based determination technique.

19. The test meter of claim 11 wherein the signal processing module is configured to receive the first signal and the second signal in a sequential manner.

20. The test meter of claim 11 wherein the signal processing module is configured to receive the first signal and the second signal in a simultaneous manner.

21. A method for determining multiple analytes in a single bodily fluid sample, the method comprising:
inserting a co-facial multi-analyte test strip into a test meter such that:
a first electrical connector of the test meter comes into contact with a first analyte contact pad of a first working electrode of the multi-analyte test strip;
a second electrical connector of the test meter comes into contact with a counter/reference electrode contact pad of a shared counter/reference electrode of the multi-analyte test strip; and
a third electrical connector of the test meter comes into contact with a second analyte contact pad of a second working electrode of the multi-analyte test strip;
determining a first analyte and a second analyte in a single bodily fluid sample applied to the co-facial multi-analyte test strip using a signal processing module of the test meter,
wherein, during the determining step, the signal processing module receives a first signal via the first electrical connector and the second electrical connector and employs the first signal for the determination of the first analyte; and
wherein, during the determining step, the signal processing module receives a second signal via the second electrical connector and the third electrical connector and employs the second signal for the determination of the second analyte; and
wherein the first working electrode and second working electrode are disposed on a first insulating layer of the co-facial multi-analyte test strip in a planar inline configuration; and
wherein the shared counter/reference electrode layer is configured in an opposing relationship to the first working electrode and the second working electrode.

22. The method of claim 21 wherein the first analyte is non-identical in comparison to the second analyte.

23. The method of claim 22 wherein the first analyte is glucose.

24. The method of claim 23 wherein the second analyte is a ketone.

25. The method of claim 24 wherein the second analyte is 3-hydroxybutyrate.

26. The method of claim 21 wherein the bodily fluid sample is a whole blood sample.

27. The method of claim 21 wherein the signal processing module is configured for determination of the first analyte and determination of the second analyte via an electrochemical-based determination technique.

28. The method of claim 21 wherein the determining step includes the signal processing unit receiving the first signal and the second signal in a sequential manner.

29. The method of claim 21 wherein the determining step includes the signal processing receiving the first signal and the second signal simultaneously.

30. The method of claim 21 wherein the bodily fluid sample is applied to the co-facial multi-analyte test strip prior to the inserting step.
